(19) United States
(12) Patent Application Publication
(10) Pub. No.: US 2008/0318317 A1
(43) Pub. Date: Dec. 25, 2008

(54) Kit for Preparing a Composition Comprising Fat Cells

(75) Inventors: Regis Roche, La Montagne De La Reunion (FR); Franck Festy, La Montagne De La Reunion (FR)

Correspondence Address: YOUNG & THOMPSON, 209 Madison Street, Suite 500, ALEXANDRIA, VA, 22314, US

(73) Assignees: FRANCK FESTY, LA MONTAGNE DE LA REUNION (FR); UNIVERSITE DE LA REUNION, SAINT DENIS DE LA REUNION (FR); REGIS ROCHE, LA MONTAGNE DE LA REUNION (FR)

(21) Appl. No.: 12/067,493

(22) PCT Filed: Sep. 20, 2006
(86) PCT No.: PCT/FR2006/050913

§ 371 Date: Aug. 29, 2008

(30) Foreign Application Priority Data
Sep. 20, 2005 (FR) 0552827

(51) Int. Cl. C12N 5/06; C12M 1/00

(52) U.S. Cl. 435/378; 435/308.1

(57) ABSTRACT

A kit for preparing a composition including fat cells includes: a) at least one sterile tube (1) adapted to be centrifuged, including a body (11) and a plug (12) including an element made of elastic and retractable material, the body (11) being hermetically sealed by the plug (12), the plug (12) including an air outlet (13) provided with a filter membrane having a size of pores of less than 0.30 μm; and b) at least one sterile needle including a central channel, one end of which is provided with a device for being fixed to a syringe, and the other end of which is bevelled, the diameter of the central channel being not less than 3 millimeters, preferably not less than 5 millimeters.

The field of the present invention relates to the way to obtain fat cells and to apply the resulting cell-based compositions to prepare medically or aesthetically dedicated compositions intended to locally regenerate tissues, amongst which adipose tissues.

Many methods for obtaining adipocyte precursor cells are known from the state of the art.

Generally, such methods comprise as a common characteristic a step for obtaining a raw adipose tissue resulting from a lipoaspirate, followed with an adipose tissue enzymatic digestion step so as to release fat cells from the surrounding connective tissue, then with an adipocyte precursor cell purification step.

Generally, the adipocyte precursor cells are then used for their ability as stem cells to differentiate into a plurality of distinct cell types, to be used in therapy.

Such a method is for example described in the American patent application No US 2003/0,082,152 issued 1 May 2003. According to this American patent application, adipocyte precursor cells, once purified, are cultured for varying time periods in the presence of different soluble factors, so as to differentiate into cells of various types. For example, for inducing the differentiation of adipocyte precursor cells into osteogenic tissue, they are incubated together with a combination of dexamethasone, ascorbic acid phosphate and beta-glycerophosphate. For inducing the differentiation of adipocyte precursor cells into chondrogenic tissue cells, the precursor cells are incubated in the presence of foetal calf serum, TGF-beta 1 and insulin. For inducing the differentiation of adipocyte precursor cells into myogenic tissue cells, the purified adipocyte precursor cells are incubated in the presence of equine serum and hydrocortisone. For inducing the differentiation of adipocyte precursor cells into adipogenic tissue, the purified adipocyte precursor cells are incubated in the presence of a combination of isobutyl-methylxanthine, dexamethasone, insulin and indometacin.

According to other methods, the adipocyte precursor cells, after having been submitted to a purification step, are administered to patients without implementing any pre-differentiation step.

Such methods for producing adipocyte precursor cells comprise an adipocyte precursor cell purification step which may have a varying duration and be more or less complicated. Said purification step may consist either in a step for positively selecting adipocyte precursor cells, or in a negative selection step wherein non desired cells are discarded or in a combination of a positive and a negative selection step. Carrying out positive selection steps and/or negative selection steps in a method for preparing a cell fraction enriched with adipocyte precursor cells was for example described in the American patent application No US 2004/0,106,196 issued 3 Jun. 2004.

In the American patent application No US 2004/0,106,196, a step is described for example for positively selecting adipocyte precursor cells by conducting a precursor cell selective adhesion, using for example various filtration membrane support types. Antibodies may also be used, as well as combinations of antibodies that do recognize surface molecules expressed on precursor cells, or on the contrary on the other cells within the start cell population. Such antibodies are fixed on a solid support, so as to selectively retain cells which do express on their surface the antigens against which said fixed antibodies are directed. The purification of adipocyte precursor cells may also be effected by immunomagnetic separation (IMS) using the suitable antibodies. Also, the purification step may be effected by implementing a first step of cell incubation using antibodies directed against cell surface antigens, then transferring the thus treated cells to an immunochromatographic column onto which are immobilized those secondary antibodies that do fix onto the previously mentioned primary antibodies. The cell purification step may also be effected by implementing continuous or discontinuous density gradients. The adipocyte precursor cell purification step may also be conducted using, cell-sorting devices such as elutriation devices, with or without any counter-flow. Purification of adipocyte precursor cells may also be obtained by conducting on the plastic surface of a culture substrate a selective adhesion step with the start cell population.

After purification, the purified adipocyte precursor cells as hereinabove described may be directly administered to the patients.

According to the objectives that are here aimed at, the hereinabove mentioned methods of the state of the art may reveal fully satisfying, especially for preparing cell populations differentiated into various tissue types or when envisaged therapeutic applications do require adipocyte precursor cells with a very high degree of purity.

However, the methods for preparing adipocyte precursor cells described from the state of the art, and which for most of them are illustrated above, are excessively long and expensive and are thus intended to remedy to physiologic conditions, and especially pathological conditions, that might threaten the patient's life.

Yet there is a need in the state of the art for methods for preparing adipocyte precursor cells which are less complicated, faster to implement and by far less expensive as compared to the known methods.

According to the present invention, a method for preparing a fat cell-enriched cell fraction was developed, including an adipocyte precursor cell-enriched fraction and a mature fat cell-enriched fraction, which is less complicated and less expensive as compared to the known methods, as well as a kit specifically designed for implementing this new method.

As used herein, “fat cells” or “adipocytes” are intended to include precursor and mature fat cells.

As used herein, “precursor cells” or “adipocyte precursor cells” are intended to mean stem cells contained within adipose tissue, which are able under suitable conditions to differentiate into cells of various cell types, such as previously described from the state of the art.

According to the present invention, said “precursor cells” do include multipotent and pluripotent cells that can differentiate into a plurality of cell types, including adipogenic, chondrogenic, cardiogenic, dermatogenic, haematopoietic, hemangiogenic, myogenic, nephrogenic, urogenitogenic, osteogenic, pericardiogenic or stroma cells.

The applicant focused on developing a kit to be used in the preparation of a composition comprising fat cells which enables to implement an uncomplicated method for preparing such a composition, to be used routinely by the practitioner without requiring any expensive instrumentation.

The applicant thus strove to develop a kit for preparing a composition comprising purified fat cells in order to allow the practitioner to use the purified fat cell-containing final composition, whenever required extemporaneously, in human or animal subjects.

During this research, the applicant did develop a sterile tube, adapted to be centrifuged, and which allows for a repeated use; under sterile conditions, and if possible under non-pyrogenic conditions as well, during a process for preparing a composition comprising fat cells, which may be used extemporaneously in human or animal subjects.

It is therefore an object of the present invention to provide a sterile tube (1) adapted to be centrifuged, including a body (11) and a plug (12) comprising an element made of elastic and retractable material, the body (11) being hermetically sealed by the plug (12), said plug (12) comprising an outlet (13) allowing gas exchanges between the inside of the tube and the outer environment to proceed, said outlet (13) being provided with a filter membrane having a size of pores of less than 0.30 μm.

The various additional features that may characterize the above mentioned tube (1) will be described hereafter in accordance with the features of the kit the tube (1) does belong to.

It is also an object of the present invention to provide a kit for preparing a composition comprising purified adipocyte precursor cells to be used extemporaneously in human or animal subjects, said kit comprising:
a) at least one sterile tube (1) adapted to be centrifuged, including a body (11) and a plug (12) comprising an element made of elastic and retractable material, the body (11) being hermetically sealed by the plug (12), said plug (12) including an air outlet (13) provided with a filter membrane having a size of pores of less than 0.30 μm;
b) at least one sterile needle comprising a central channel, one end of which is provided with a device for being fixed to a syringe, and the other end of which is bevelled, the diameter of the central channel being not less than 3 millimeters, preferably not less than 5 millimeters.

Thanks to the hereinabove kit, it is possible for the one skilled in the art, most of the time within less than two hours after having collected a lipoaspirate or haying performed a lipectomy on a patient, to obtain a composition comprising purified adipocyte precursor cells, that can be directly used for injection to the same patient, in particular, aiming at body reconstruction or therapeutic objectives.

As used herein, a “lipoaspirate” is intended to mean a tissue sample resulting from a liposuction operation.

As used herein, a “lipectomy” is intended to mean a tissue sample, preferably an adipose tissue sample, said tissue sample containing fat cells, said tissue sample being collected on a patient by means of a surgical operation.

The hereinabove kit is especially suitable for implementing a method for preparing a composition comprising purified fat cells, said method including the following steps of:
a) introducing an adipose tissue suspension resulting from a lipoaspirate into a tube (1) such as previously defined;
b) allowing an enzymatic digestion of the adipose tissue suspension to proceed in the tube (1) using an enzyme-based preparation containing at least one protease;
c) centrifuging the suspension after the enzymatic digestion;
d) collecting the centrifuged suspension fractions containing the interesting cells:
   d1) collecting the adipocyte precursor cell (AP) fraction, which is concentrated in the pellet located in the bottom portion of the tube (1);
   d2) if necessary, collecting the mature adipocyte (MA) fraction located in the upper part of the adipose tissue suspension centrifuged in step c);
e) incubating the (AP) fraction in a hypotonic solution so as to lyse erythrocytes; and
f) preparing a fat cell composition from one of the following cell fractions f1), f2) or f3):
   f1) the (AP) fraction of adipocyte precursor cells such as obtained at the end of step e);
   f2) the (MA) fraction of mature adipocytes such as obtained in step d1); and
   f3) a mixture comprising (AP) fraction cells and (MA) fraction cells.

The compositions obtained using the method of the invention may be used extemporaneously in human or animal subjects.

In particular, the kit of the invention is especially suitable for implementing methods for preparing precursor cell-enriched fractions without performing any complicated step of adipocyte precursor cell high level purification, apart from steps c), d) and e) of the general method as described hereinabove.

As already stated, the kit of the invention comprises at least one sterile tube (1) such as previously defined, which is sterile and ready-to-use to perform steps a) to d), or even steps a) to e) of the above method.

For example, the tube (1) may be used to step e), or even in some cases to step f), of the hereinabove defined general method when, once the suspension treated with the enzyme-based preparation has been centrifuged, the near totality of the centrifuged, enzymatically treated adipose tissue suspension is discarded while retaining the cell pellet located in the bottom portion of the tube (1) which contains the precursor cells, said cell pellet being resuspended in step e) with the hypotonic solution, prior to conducting one or more centrifugation and washing step(s) using an isotonic medium, then the precursor cells are combined with the biological matrix material, in step f) of the present method.

To allow steps a) to d), or alternately steps a) to e), of the above method to be conducted with just the sterile tube (1), said sterile tube (1) comprises a plug (12) comprising an element made of elastic and retractable material so that said plug (12) can be punctured many times by the needle of a syringe, while preserving its tightness against the outer environment after the needle withdrawal.

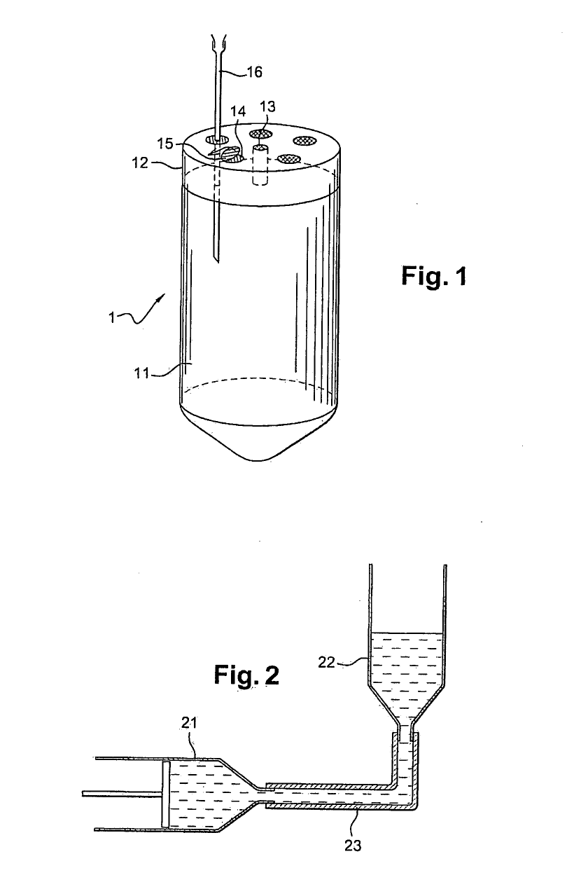

According to a first alternative, the whole plug (12) is made of an elastic and retractable material, as illustrated in FIG. 1.

According to a second alternative, only a part of the plug (12) is made of an elastic and retractable material, preferably the central portion of the plug (12). According to this alternative, the central portion of the plug (12) which is made of an elastic and retractable material, is introduced, for example is crimped or otherwise set, in the outer portion of the plug (12). The outer portion of the plug (12) which may be of metal or plastic, is in contact with the body (11) upper edge of the tube (1), and serves as sealing element for the tube (1).

The plug (12) may be screwed to the tube (1), thanks to additional screw threads located respectively on the plug (12) and on the body (11) upper end of the tube (1). In other embodiments, the plug (12) is forced into the body (11) upper end of the tube (1).

In particular; the plug (12) elastic and retractable material makes it possible to pierce through the plug with the needle of a syringe, at least in the following steps of the present general method such as hereinabove:
in step a) when introducing the adipose tissue suspension into the tube (1);
if necessary, in step b) when introducing into the tube (1) an enzyme-based preparation, in the embodiments of the present method wherein the tube (1) does not initially contain such an enzyme-based preparation, in a liquid or a lyophilized form;
in step d) wherein the various cell fractions of the interesting cell-containing centrifuged suspension are collected, whenever required by removing the cell suspension volume located in the tube (1) above the pellet located in the bottom portion of the tube containing the (AP) precursor cell fraction;
in step e) wherein the hypotonic solution may be directly added to the tube (1) so as to resuspend the cell fraction comprising the precursor cells;
during the step(s) of washing and centrifugating the cell fraction obtained at the end of step e) with an isotonic medium, in order to remove the erythrocytes which were lysed as a consequence of the previous incubation using the hypotonic medium; and
if necessary, during step f) for combining adipocyte precursor cells with the biological matrix material, during which step the biological matrix material may be directly added to the tube (1) that contains the cell suspension in an isotonic medium.

FIG. 1 illustrates a particular embodiment of the tube (1) included in the kit of the invention.

In this advantageous embodiment, the plug (12) of the sterile tube (1) comprises at least one sterile puncture area (14). The sterility of the puncture area (14) is maintained in the long run thanks to a strippable film (15) which covers the puncture area (14). The strippable film (15) is made of a liquid-tight and optionally also gas-tight material. For example, the strippable film (15) may consist in a paraffin film or in a metal film, for example an aluminium film. If necessary, the strippable film (15) may be withdrawn, and then applied again onto the corresponding puncture area (14) a number of times, so as to enable a repeated use of the puncture area (14) for the needle of a syringe going through in the different steps of adding or collecting material to or from the tube (1), provided that the conditions of sterility upon adding or collecting the material to or from the tube (1) are rigorously met.

However, advantageously, the plug (12) of the sterile tube (1) comprises multiple puncture areas (14), in particular at least two puncture areas (14), for example from two to five puncture areas (14), so as to implement the hereinabove defined general method by using only one time the same puncture area (14) for adding or collecting material to or from the tube (1).

In another embodiment of the sterile tube (1) plug (12), the latter may comprise more than five puncture areas (14). Thus, in some embodiments of the kit of the invention, the plug (12) of the sterile tube (1) does comprise up to ten puncture areas (14).

Generally, the tube (1) comprises a tube the body (1) of which is made of a plastic material, for example polypropylene, polystyrene or polyethylene, of a known type that is well adapted to cell suspension centrifugation. The tube (1) may have a volume ranging from 50 milliliters to 100 milliliters. The tube (1) is adapted to a centrifugation step at an acceleration of at least 1000 g, preferably of at least 1500 g.

Generally speaking, the tube (1) is a sterile and non-pyrogenic tube.

As already stated, the plug (12), or at least part of it, is made of an elastic and retractable material of any type known to the one skilled in the art, such as for example a rubber or a silicone material.

According to a further characteristic, the plug (12) comprises an air outlet (13) provided with a filter membrane having a size of pores of less than 0.30 μm. The air outlet (13) does allow for gas exchange between the inside of the tube (1) and the outer environment to proceed, while preventing particles, including microorganisms, from entering the tube (1). The air outlet (13) makes it possible to easily add or collect some material to or from the tube (1), without simultaneously causing, a vacuum or an overpressure inside the tube (1).

The filter membrane is of a type known to the one skilled in the art, for example a nitrocellulose filter. The filter membrane with which the air outlet (13) is fitted may have a pore size of about 0.2 μm, for example of 0.22 μm.

In some embodiments of the kit of the invention, the sterile tube (1) contains an enzyme-based preparation suitable for adipose tissue digestion, said enzyme-based preparation being either in a liquid or in a solid form, for example in the form of a lyophilized enzyme-based preparation. Most preferably, said enzyme-based preparation is sterile and non-pyrogenic. According to this embodiment of the kit of the invention, step b) of the hereinabove defined general method may be carried out immediately after introduction of the adipose tissue suspension into the tube (1).

In other embodiments of the kit of the invention, the sterile tube (1) does not comprise any enzyme-based preparation. In this other embodiment of the kit of the invention, said kit may further comprise a container, for example a tube or a flask, containing an enzyme-based preparation suitable for adipose tissue digestion, said enzyme-based preparation being either in a liquid or in a lyophilized form.

Said container is a sterile, non-pyrogenic and hermetically sealed container. It may come as a plastic or a glass flask or as a glass ampoule or even as a flexible bag containing the enzyme-based preparation, preferably in a liquid form.

Advantageously, the enzyme-based preparation comprises at least one protease, and preferably at least one collagenase of any type known to the one skilled in the art. As an illustration, the one skilled in the art may employ an enzyme-based preparation as described in the U.S. Pat. No. 5,952,215 or in the U.S. Pat. No. 6,475,764.

As previously described, the kit of the invention further comprises at least one sterile bevel needle to be suitably adapted to the syringe outlet, the central channel diameter of which is at least 3 millimeters, preferably at least 5 millimeters. This sterile and non-pyrogenic needle is used in step a) of the hereinabove general method for introducing into the tube (1) the adipose tissue suspension that was previously collected in a sterile manner from the patient.

Advantageously, this large-diameter needle comprises a bevelled end so as to prevent any “punching effect” when piercing through the plug (12) of the tube (1) in use in step a) of the hereinabove general method. This sterile and non-pyrogenic large-diameter needle should have a sufficient length to go through the plug of the tube. Advantageously, this sterile and non-pyrogenic needle has a length of not less than 50 millimeters.

The other end of the sterile and non-pyrogenic large-diameter needle, is provided with a device for being fixed to a syringe, which may be a fastening device of any type known to the one skilled in the art. Advantageously, said device for being fixed to a syringe comprises a locking system of the “Luer-Lock®” type well known to the one skilled in the art.

In some embodiments of the kit of the invention, said kit comprises several sterile and non-pyrogenic tubes (1). Indeed, the adipose tissue collection volume does frequently exceed the working volume of a single sterile tube (1) which requires the use in step a) of the hereinabove present general method, of a plurality of sterile tubes (1). Advantageously, a kit of the invention includes 2, 3, 4, 6, 7 or 8 sterile tubes (1).

In some embodiments of the kit of the invention, said kit further comprises at least one flask containing a hypotonic medium suitable for erythrocyte lysis. The hypotonic medium-containing flask, which may be of any type known to the one skilled in the art is advantageously used for carrying out step e) of the present general method as defined hereinabove.

As an illustration, said hypotonic medium is composed of an aqueous solution comprising (i) potassium hydrogencarbonate (KHCO₃) to a final concentration of 10 mM, (ii) ammonium chloride (NH₄Cl) to a final concentration of 155 mM and (iii) ethylene diamine tetraacetic acid disodium salt (Na₂ EDTA) to a final concentration of 1 mM. Advantageously, said hypotonic medium has a pH value ranging from 7.2 to 7.6.

In other embodiments according to the present invention, said kit also comprises at least one flask containing an isotonic medium suitable for cell resuspension, for example during the washing and centrifugation step(s) that may be carried out after erythrocyte lysis step e), and prior to combining the purified adipocyte precursor cell fraction with the biological matrix material.

Advantageously, the hypotonic medium-containing flask may be a flexible plastic bag, for example a Ringer-type flexible plastic bag of 250 ml, comprising a silicone sealed plug which can be pierced by a needle. The plug, for example made of silicone, should be pierceable, preferably many times by means of a 18 gauge needle without affecting the tightness of the sterile and non-pyrogenic hypotonic solution-containing flask. In some embodiments, the hypotonic solution-containing plastic bag is set in an easy-to-open individual sterile packaging. According to other embodiments of the kit of the invention, said kit additionally comprises at least one sterile and non-pyrogenic filtering device provided with a filter membrane having a size of pores ranging from 40 μm to 200 μm.

The sterile filtering device is used in some embodiments of the general method as previously defined, wherein the cell suspension obtained at the end of the washing and centrifugation steps after step e) of such method is introduced into the inner volume of a syringe by suction, then filtered once the filtering device has been set in place on the syringe, tip, so as to make the cell suspension free from any debris; in particular connective tissue debris, in order to retrieve, after filtration, a suspension containing; exclusively or almost exclusively the interesting cells.

The filtering device of the kit of the invention is of any known type. Advantageously, the filter membrane having a size of pores ranging from 40 μm to 200 μm, consists in a nylon filter membrane which is fixedly set in place inside the filtering device. The filtering device itself is of any known type, for example such as those filtering devices which can be adapted to syringes traditionally marketed by the MILLIPORE or SARTORIUS companies. Advantageously, the filtering device comprises an adaptor system to the syringe hub composed of a “Luer-Lock®” type locking means. Advantageously, the kit of the invention further comprises a needle that can be adapted to the other end of the filtering device, whenever required provided with a “Luer-Lock®” type locking system, so as to introduce the adipocyte precursor cell suspension into a receiving tube, advantageously a sterile tube (1).

In some embodiments of the kit of the invention, the filter and the needle together form an integral part, the needle; being indeed directly welded to the filtering device.

Preferably, the needle intended to be used in combination with the filtering device is a needle of not less than 16 Gauges, and the size of which is sufficient to go through the plug of the tube (1), the needle being preferably at least 50 millimeters long.

In this particular embodiment of a kit of the invention, said kit may comprise a set comprising a combination of a filtering device and a needle such as previously defined in a plurality of units.

Advantageously, a kit of the invention includes 2, 3, 4, 5, 6, 7, 8 units of a set comprising the combination of a filtering device and a needle such as previously defined.

Generally speaking; for implementing the present general method as defined hereinabove, there are as many sets comprising the combination of a filtering device and a needle as sterile tubes (1) used in step a) of the present method.

In yet other embodiments of the kit of the invention, said kit further comprises at least one needle of not more than 18 Gauges and preferably at least 125 millimeters long. Said needle is suitable for carrying out step d1) of the present method, to recover the centrifugation supernatant that is free from any (AP) adipocyte precursor cell-enriched fraction. Said needle is also suitable for removing the liquid staying within the tube (1) under the cell band containing the mature adipocytes prior to retrieving the precursor cells which are in the form of a cell pellet in the bottom portion of the tube (1).

In yet other embodiments of the kit of the invention, said kit further comprises a device for preparing a fat cell final composition with a matrix material, for carrying out step f) of the general method as defined in the present description.

Thus, in some embodiments of the method of the present invention, the final composition is prepared in step f) by combining a matrix material with the fat cells, that is to say with the (AP) cells, the (MA) cells or a mixture of both.

As used herein, a “matrix material” is intended to mean a non cytotoxic material which is sterile and non-pyrogenic and promotes the implantation of the fat cells as obtained by the method, in the body of a human or an animal subject.

Such a device (2) is illustrated in FIG. 2:
(i) a first syringe (21) intended to be filled with a suitable volume of a matrix material;
(ii) a second syringe (22) intended to be filled with a suspension of adipocyte precursor cells in an isotonic medium; and
(iii) a pipe (23) connecting the outlet of each syringe (21; 22) said pipe (23) putting the syringes (21) and (22) into fluid communication with each other.

Advantageously, the syringes (21) and (22) are sterile and non-pyrogenic plastic syringes of a known type. Advantageously, the syringes (21) and (22) are syringes with a working volume of at least 50 milliliters and which may extend up to 100 milliliters or more.

Advantageously, the pipe (23) is a sterile and non-pyrogenic, flexible or rigid pipe. The pipe (23) may be made of a flexible plastic material or of silicone. In a particular embodiment of the device (2), the first syringe (21) is pre-filled with a suitable volume of the selected matrix material.

According to a further additional characteristic of the kit of the invention, said kit may also comprise at least one tube rack in plastic suitable for receiving tubes (1). The tube rack may consist in a tube rack of any type known to the one skilled in the art. In particular, said tube rack may be designed very simply to be sufficient for maintaining the tubes (1) in a vertical position during the various manipulations required for implementing the general method as previously defined in the present description. Advantageously, said tube rack should have a sufficiently large size to be able to keep in a vertical position up to 20 tubes (1). Said tube rack may be of a very simple design and its production cost may be very low.

It is a further object of the present invention to provide a method for preparing a composition comprising fat cells, to be used in human or animal subjects, said method comprising the various following steps of:
a) introducing an adipose tissue suspension resulting from a lipoaspirate or a lipectomy into a tube (1), such as defined in the present description;
b) allowing an enzymatic digestion of the adipose tissue suspension to proceed in the tube (1) using an enzyme-based preparation containing at least one protease;
c) centrifuging the suspension after the enzymatic digestion;
d) collecting the centrifuged suspension fractions containing the interesting cells:
   d1) collecting the adipocyte precursor cell (AP) fraction of precursor cells in the pellet located in the bottom portion of the tube (1);
   d2) if necessary, collecting the mature adipocyte (MA) fraction recovered in the upper part of the centrifuged adipose tissue suspension;
e) incubating the AP fraction in a hypotonic solution, so as to effect an erythrocyte lysis; and
f) preparing an adipocyte composition from the following cell fractions:
   f1) the (AP) fraction of precursor cells such as obtained at the end of step e):
   f2) the (MA) fraction of mature adipocytes such as obtained in step d1); and
   f3) a combination of cells of the (AP) fraction and cells of the (MA) fraction.

Prior to step a) of the present method, some adipose tissue is collected in a sterile manner, by liposuction according to techniques known to the one skilled in the art, either by manual suction, or by using an adapted pump.

Collected adipose tissue is recovered in a sterile syringe, for example in a sterile syringe of the type provided with a “Luer-Lock®” type screw locking system. After settling, the serum is removed from the syringe.

To continue the present method, the one skilled in the art uses the kit of the invention such as previously defined in detail in the present description.

### Step a) of the Method

When using the kit of the invention, the sterile needle representing element b) of the kit, is adapted on the adipose tissue-containing syringe. The contents of the syringe are then introduced into the tube (1).

In some cases, prior to step a) of the present method, several syringes containing adipose tissue are obtained after liposuction. In step a) of the present method, the contents of several syringes containing adipose tissue may be introduced into a single tube (1).

If necessary, when the adipose tissue volume does exceed the working volume of the tube (1), several tubes (1) included, in a kit of the invention may be used to recover adipose tissue.

### Step b) of the Method

When using the embodiment of the kit of the invention wherein the tube (1) is pre-filled with an enzyme-based preparation suitable for adipose tissue digestion, step b) of the enzymatic digestion may be directly carried out.

On the contrary, in the embodiment of the kit of the invention wherein the tube (1) does not contain any enzyme-based preparation, step b) of the enzymatic digestion proceeds after having introduced into the tube (1) the suitable amount of the enzyme-based preparation included in the kit in a separate container.

Advantageously, in step b) of the present method, the enzyme-based preparation comprises collagenase which is present at a final concentration in the tube (1) of from 0.05 to 5 collagenase units per ml of adipose tissue contained in the tube (1), the collagenase unit being the activity unit PZ such as defined by Wünsch. According to Wünsch, one collagenase unit does catalyse hydrolysis of 1 μmole of 4-phenylazobenzyloxycarbonyl-L-prolyl-L-leucyl-glycyl-L-prolyl-D-arginine per minute at 25° C. and pH value 7.1.

When the enzyme-based preparation suitable for adipose tissue digestion is in the form of a powder, for example in a lyophilized form, in a separate container of the tube (1), the one skilled in the art reconstitutes an enzyme-based preparation liquid solution by adding to said container a suitable volume of an isotonic medium compatible with the human use, for example a Ringer-Lactate type isotonic medium, for the enzymes be in solution again. A defined volume of the enzymatic solution is then introduced by injection into the adipose tissue-containing tube(s) (1).

Advantageously, once the adipose tissue has been combined with the enzyme-based preparation in the one or more tube(s) (1), a human-compatible physiological medium is added to each tube (1) until they are filled therewith.

The tube(s) (1) is or are then incubated at a temperature that may vary from 10° C. to 60° C., more preferably from 25° C. to 45° C., even more preferably from 30° C. to 40° C., for example at 37° C., so as to carry out enzymatic digestion step b). During step b), the tubes (1) are preferably continuously stirred. Generally, in step b), the enzymatic digestion time does range from 5 minutes to 2 hours, more preferably from 10 minutes to 30 minutes.

Optionally, at the end of step b), the suspension obtained after the enzymatic digestion may be filtered through a filtering device, preferably a filtering device of a here previously described type, provided with a filter membrane having a size of pores ranging from 40 μm to 200 μm. This optional filtration step makes it possible to remove adipose tissue debris and wastes that do result from the enzymatic digestion, to obtain at the end of step b) a liquid suspension containing essentially cells.

### Step c) of the Method

In step c) of the present method, the cell suspension, if necessary cleared of the most of adipose tissue debris by filtering, is centrifuged at an acceleration of from 10 g to 5000 g, more preferably of from 800 g to 3000 g, even more preferably from 1000 g to 2000 g.

The centrifugation step c) time does range from 5 seconds to 30 minutes, depending on the selected centrifugation speed. As an illustration, the centrifugation may be conducted at 1500 g for 10 minutes.

At the end of the centrifugation step c), the precursor cell-enriched cell fraction, also called (AP) fraction in the present description, is recovered in the pellet located in the bottom portion of the tube (1).

At the end of the centrifugation step c), a mature adipocyte-enriched cell fraction is recovered in the form of a cell band located in the upper part of the liquid suspension contained in the tube (1).

### Step d) of the Method

Step d1) of the Method

In step d1) of the present method, the (AP) fraction enriched with precursor cells is recovered by removing the supernatant liquid located in the tube (1) over the cell pellet. Advantageously, the supernatant is removed from the tube (1) by suction by means of a syringe provided with a sterile needle the length of which does correspond at least to the total height of the tube (1).

Step d2) of the Method (Optional).

In some embodiments of the method of the present invention, the mature adipocyte fraction, also called (MA) fraction in the present description, is also recovered in step d2), which fraction was collected from the upper part of the centrifuged adipose tissue suspension.

Recovering the mature adipocyte-containing cell band may be effected by means of a syringe provided with a sterile and non-pyrogenic needle, by suction of the interesting cell band into the syringe.

Generally, the mature adipocyte (MA) fraction is then introduced into an empty tube (1).

In practice, when step d2) has been performed, this step typically occurs prior to step d1).

### Step e) of the Method

Following step d1), the (AP) fraction of precursor cells is resuspended in a sterile and non-pyrogenic hypotonic medium, which is introduced into the tube (1) containing the cell pellet resulting from step d1), by means of a syringe provided with a sterile and non-pyrogenic needle.

After having introduced a suitable volume of hypotonic medium, the tubes (1) containing the precursor cells in the hypotonic medium are vigorously stirred, then centrifuged. Advantageously, the tubes (1) are centrifuged at an acceleration of from 100 g to 5000 g for a time period ranging from 30 seconds to 30 minutes. The conditions of centrifugation at the end of step e) of the present method are generally the same as those of the centrifugation in step c) of the present method.

At the end of the hereinabove centrifugation, the precursor cells are retrieved in the pellet in the bottom portion of the tubes (1). The supernatant liquid is removed from the tube by suction, with a syringe provided with a sterile and non-pyrogenic needle.

The cell pellet of each tube (1) is then resuspended in a human injection-compatible isotonic medium, such as for example, a Ringer-Lactate type isotonic medium. The isotonic medium is introduced into the tubes (1) by means of a sterile syringe provided with a sterile and non-pyrogenic needle.

If necessary, the thus obtained cell suspensions are brought together in a single tube (1), if the volume allows it.

Optionally, the thus resuspended cell fraction may be submitted to a filtration step using a filtering device of a here previously described type provided with a filter membrane having a size of pores ranging from 40 μm to 200 μm, so as to remove from the cell fraction the unwanted adipose tissue or connective tissue wastes that may be present within the cell suspension.

In the embodiments of the method of the present invention, wherein step d2) is carried out, mature adipocyte-containing cell fractions do undergo a centrifugation of the same type as that described hereinabove for the cell fraction resulting from step d1), then they are resuspended in a human injection-compatible isotonic medium, for example of a Ringer-Lactate type.

Generally, speaking, it is equally recommended for the precursor cells resulting from step d1) as for the mature adipocytes resulting from step d2) to perform several centrifugation steps, then to resuspend the cell fractions in an isotonic medium (washing) before collecting the final precursor cell (AP) fractions or the final mature adipocyte (MA) fractions, respectively, to be used for carrying out step f) of the present method.

### Step f) of the Method

In step f) of the present method, the final composition comprising (AP) fraction cells, (MA) fraction cells or combinated cells from the (AP) fraction and the (MA) fraction, for example in a cell ratio (AP):(MA) ranging from 1:99 to 99:1, is accomplished whenever required in combination with one or more pharmaceutical active agent(s) and/or one or more pharmaceutically acceptable excipient(s). For example, fat cells may be combined with a matrix material, so as to obtain a purified fat cell-containing final composition. Such a composition may be used extemporaneously and be directly administrated to human or animal subjects.

The matrix material may be composed of a biological matrix material, which may be selected from any biological matrix materials compatible with an administration to human or animal subjects, of any type known to the one skilled in the art. Advantageously, said biological matrix material is selected from resorbable matrix materials, which include collagen, hyaluronic acid or hydrogels such as acrylic hydrogels also optionally containing hyaluronic acid.

As an illustration, a collagen-containing resorbable matrix material may be selected from materials marketed under the trade names Artecoll®, Zyderm®, Zyplast® or Autologen®.

As an illustration, a hyaluronic acid-containing resorbable matrix material may be selected from materials marketed under the trade names Hylaform®, Restyiane®, Perlahe®, Juvederm®, Hylan® or Hydrafill®.

As an illustration, a resorbable matrix material based on hyaluronic acid-containing acrylic hydrogel may be selected from materials marketed under the trade names Derlakuve® or Dermadeep®.

The matrix material may also be selected from non-resorbable matrix materials which include natural or synthetic polymer-based materials such as expanded polytetrafluorethylene, expanded polyester, poly-L-lactic acid, crosslinked polyacrylamides or polyalkylimide.

As an illustration, an expanded polytetrafluoroethylene-based non-resorbable matrix material may be selected from materials marketed under the trade names Softform® or Gortex®.

As an illustration, a resilient expanded polyester-based non-resorbable matrix material may be selected from materials marketed under the trade names M-SI Fil® or Filladerm®.

As an illustration, a poly-L-lactic acid-based non-resorbable matrix material, for example comprising poly-L-lactic acid microspheres, may be a material marketed under the trade name New File®.

As an illustration, a crosslinked polyacrylamide-based non-resorbable matrix material may be a material marketed under the trade name Aquamid®.

As an illustration, a polyalkylimide-based non-resorbable matrix material may be a material marketed under the trade name Bio-Alcamid®.

As an illustration, hyaluronic acid crosslinked or not may be used as a biological matrix material.

Advantageously, in step f) of the present method, the final composition containing ready-to-use adipocyte precursor cells for administrating to human subjects comprises an amount of matrix material ranging from 0.001 to 10 g of said material for a volume of 100 milliliters of said final composition. Preferably, the ready-to-use final composition comprises from 0.1 to 1 g of biological matrix material, for a volume of 100 milliliters of said final composition.

In step f) of the present method, the fat cell final composition may in some embodiments contain an active agent or a combination of pharmaceutical active agent(s) for human or animal use.

As an illustration, the fat cell final composition may comprise one or more growth factor(s), such as for example the fibroblast growth factor (FGF), the epidermal growth factor (EGF) or a colony stimulating factor (CSF).

As an illustration, a ready-to-use final composition according to the present invention comprises from 0.2 to 0.5 g of non crosslinked hyaluronic acid per 100 ml of the final composition.

Generally speaking, the ready-to-use final composition according to the present invention comprises from 10³ to 10⁹ interesting cells per ml of the final composition, more preferably from 10⁴ to 10⁸ interesting cells per ml of the final composition and even more preferably from 5 to 10×10⁶ interesting cells per ml of the final composition.

To conclude, it is an object of the present invention to provide a sterile tube (1) such as defined in detail in the present description.

1. A kit for preparing a composition comprising fat cells, said kit comprising:
a) at least one sterile tube (1) adapted to be centrifuged, including a body (11) and a plug (12) comprising an element made of elastic and retractable material, the body (11) being hermetically sealed by the plug (12), said plug (12) including an air outlet (13) provided with a filter membrane having a size of pores of less than 0.30 μm;
b) at least one sterile needle, comprising a central channel, one end of which is provided with a device for being fixed to a syringe, and the other end of which is bevelled, the diameter of the central channel being not less than 3 millimeters, preferably not less than 5 millimeters.

2. A kit according to claim 1, characterized in that the plug (12) of the sterile tube (1) comprises at least one sterile puncture area (14) covered with a strippable film (15).

3. A kit according to claim 2, characterized in that the plug (12) of the sterile tube (1) comprises from 2 to 5 puncture areas (14).

4. A kit according to claim 2, characterized in that the plug (12) of the sterile tube (1) comprises more than 5 puncture areas (14).

5. A kit according to claim 1, characterized in that the sterile tube (1) contains an enzyme-based preparation suitable for adipose tissue digestion, said enzyme-based preparation being either in a liquid or in a lyophilized form.

6. A kit according to claim 1, characterized in that the sterile tube (1) does not comprise any enzyme-based preparation.

7. A kit according to claim 6, characterized in that it comprises a container containing an enzyme-based preparation suitable for adipose tissue digestion, said enzyme-based preparation being either in a liquid or in a lyophilized form.

8. A kit according to claim 1, characterized in that it includes 2, 3, 4, 5, 6, 7 or 8 sterile tubes (1).

9. A kit according to claim 1, characterized in that it comprises a hypotonic medium-containing flask suitable for the erythrocyte lysis.

10. A kit according to claim 7, characterized in that said hypotonic medium has a pH value ranging from 7.2 to 7.6.

11. A kit according to claim 1, characterized in that it comprises a flask containing an isotonic medium suitable for cell resuspension.

12. A kit according to claim 1, characterized in that it comprises at least one sterile filtering device provided with a filter membrane having a pore size ranging from 40 μm to 200 μm.

13. A kit according to claim 12, characterized in that the sterile filtering device further comprises a sterile needle of not less than 16 Gauges.

14. A kit according to claim 1, characterized in that it further comprises at least one needle of not more than 18 Gauges.

15. A kit according to claim 1, characterized in that it further comprises a device (2) for obtaining a composition resulting from the combination of precursor cells with a biological matrix, said device being composed of:
(i) a first syringe (21) intended to be filled with a suitable volume of a biological matrix;
(ii) a second syringe (22) intended to be filled with a precursor cell suspension in an isotonic medium; and
(iii) a pipe (23) connecting the outlet of each syringe (21, 22), said pipe (23) putting the syringes (21) and (22) into fluid communication with each other.

16. A method for preparing a composition comprising fat cells, said method comprising following steps of:
a) introducing an adipose tissue suspension resulting from a liposuction or a lipectomy into a tube (1) such as defined in claim 1;
b) allowing an enzymatic digestion of the adipose tissue suspension to proceed in the tube (1) using an enzyme-based preparation containing at least one protease;
c) centrifuging the suspension after the enzymatic digestion;
d) collecting the centrifuged suspension fractions containing the interesting cells:
d1) collecting the adipocyte precursor cell (AP) fraction in the pellet located in the bottom portion, of the tube (1);
d2) if necessary, collecting the mature adipocyte (MA) fraction recovered in the upper part of the centrifuged adipose tissue suspension;
e) incubating the (AP) fraction in a hypotonic solution so as to lyse erythrocytes; and
f) preparing a fat cell composition from one of the following cell fractions:
f1) the (AP) fraction of adipocyte precursor cells such as obtained at the end of step e);
f2) the (MA) fraction of mature adipocytes such as obtained in step d1); and
f3) a combination of cells of the (AP) fraction and cells of the (MA) fraction.

17. A method according to claim 16, characterized in that in step f), when the combination is made with both (AP) fraction cells and (MA) fraction cells, the cell ratio (AP):(MA) does vary from 1:99 to 99:1.

18. A sterile tube (1) adapted to be centrifuged, including a body (11) and a plug (12) comprising an element made of elastic and retractable material, the body (11) being hermetically sealed by the plug (12), said plug (12) including an air outlet (13) provided with a filter membrane having a size of pores of less than 0.30 μm.

19. A sterile tube (1) according to claim 18, characterized in that the plug (12) of the sterile tube (1) comprises at least one sterile puncture area (14) covered with a strippable film (15).

20. A sterile tube (1) according to claim 18, characterized in that the plug (12) of the sterile tube (1) comprises at least two puncture areas (14).

21. A sterile tube (1) according to claim 18, characterized in that it contains an enzyme-based preparation suitable for adipose tissue digestion, said enzyme-based preparation being either in a liquid or in a lyophilized form.
